(19) United States
(12) Patent Application Publication
(10) Pub. No.: US 2020/0306347 A1
(43) Pub. Date: Oct. 1, 2020

(54) CROSSLINKED PARTICLES, COMPOSITION COMPRISING THE CROSSLINKED PARTICLES, METHOD FOR THE MANUFACTURE THEREOF, AND METHOD OF TREATING AN INFECTION

(71) Applicant: THE UNIVERSITY OF MASSACHUSETTS, Boston, MA (US)

(72) Inventors: Vincent M. Rotello, Amherst, MA (US); Ryan Francis Landis, Amherst, MA (US)

(21) Appl. No.: 16/650,719

(22) PCT Filed: Dec. 11, 2018
(86) PCT No.: PCT/US2018/064925

§ 371 Date: Mar. 25, 2020

Related U.S. Application Data
(60) Provisional application No. 62/598,070, filed on Dec. 13, 2017.

(51) Int. Cl. A61K 38/39; A61K 31/05; A61P 31/04; A61P 31/10

(52) U.S. Cl. CPC A61K 38/39; A61P 31/10; A61P 31/04; A61K 31/05

(57) ABSTRACT

Crosslinked particles including an essential oil and gelatin are described. At least a portion of the gelatin is crosslinked. A method of making the crosslinked particles includes emulsifying a mixture of an essential oil and an aqueous solution, and exposing the resulting particles to conditions effective to crosslink the gelatin to provide the crosslinked particles. The crosslinked particles and compositions including the crosslinked particles can be particularly useful in the treatment of bacterial and fungal infections.

### BACKGROUND

Bacterial biofilms are highly resilient microbial assemblies that are difficult to eradicate. See, e.g., Costerton, J. W.; Stewart, P. S.; Greenburg, E. P. Bacterial Biofilms: A Common Cause of Persistent Infections. Science 1999, 284, 1318-1322. These robust biofilms frequently occur on synthetic implants and indwelling medical devices including urinary catheters, arthro-prostheses, and dental implants. See, e.g., Lindsay, D.; von Holy, A. Bacterial Biofilms within the Clinical Setting: What Healthcare Professionals Should Know. J. Hosp. Infect. 2006, 64, 313-325; Costerton, J. W.; Montanaro, L.; Arciola, C. R. Biofilm in Implant Infections: Its Production and Regulation. Int. J. Artif. Organs 2005, 28, 1062-1068; Busscher, H. J.; Rinastiti, M.; Siswomihardjo, W.; van der Mei, H. C. Biofilm Formation on Dental Restorative and Implant Materials. J. Dent. Res. 2010, 89, 657-665. Biofilm proliferation can also occur on dead or living tissues, leading to endocarditis, otitis media, and chronic wounds. See, e.g., Costerton, W.; Veeh, R.; Shirtliff, M.; Pasmore, M.; Post, C.; Ehrlich, G. The Application of Biofilm Science to the Study and Control of Chronic Bacterial Infections. J. Clin. Invest. 2003, 112, 1466-1477; Ehrlich, G.; Veeh, R.; Wang, X.; Costerton, J. W.; Hayes, J. D.; Hu, F. Z.; Daigle, B. J.; Ehrlich, M. D.; Post, J. C. Mucosal Biofilm Formation on Middle-Ear Mucosa in the Chinchilla Model of Otitis Media. JAMA 2002, 287, 1710; James, G. A; Swogger, E.; Wolcott, R.; Pulcini, E. deLancey; Secor, P.; Sestrich, J.; Costerton, J. W.; Stewart, P. S. Biofilms in Chronic Wounds. Wound Repair Regen. 2007, 16, 37-44. The persistent infections and their concomitant diseases are challenging to treat, as biofilms develop a high resistance to host immune responses and the extracellular polymeric substances limit antibiotic penetration into biofilms. See, e.g., Stewart, P. S.; Costerton, J. W. Antibiotic Resistance of Bacteria in Biofilms. Lancet 2001, 358, 135-138; Szomolay, B.; Klapper, I.; Dockery, J.; Stewart, P. S. Adaptive Responses to Antimicrobial Agents in Biofilms. Environ. Microbiol. 2005, 7, 1186-1191. Current techniques to remove biofilms on man-made surfaces include disinfecting the surface with bleach or other caustic agents. See, e.g., Marion-Ferey, K.; Pasmore, M.; Stoodley, P.; Wilson, S.; Husson, G. P.; Costerton, J. W. Biofilm Removal from Silicone Tubing: An Assessment of the Efficacy of Dialysis Machine Decontamination Procedures Using an in Vitro Model. J. Hosp. Infect. 2003, 53, 64-71. Biofilms in biomedical contexts are very challenging, with therapies based on excising infected tissues combined with long-term antibiotic therapy, incurring high health care costs and low patient compliance due to the invasive treatment. See, e.g., Lynch, A. S.; Robertson, G. T. Bacterial and Fungal Biofilm Infections. Annu. Rev. Med. 2008, 59, 415-428. This issue is exacerbated by the exponential rise in antibiotic resistant bacteria. See, e.g., Levy, S. B.; Marshall, B. Antibacterial Resistance Worldwide: Causes, Challenges and Responses. Nat. Med. 2004, 10, S122S129.

An increase in antibiotic resistance has fueled the design and development of alternative antimicrobial agents. While a variety of platforms such as nanoparticles, polymers, metal ions, and combinations of these have been explored, these platforms often are accompanied by adverse toxicity towards mammalian cells and face practical industrial scale-up challenges that hinder their chances of reaching commercialization.

Phytochemicals have emerged as a promising alternative to traditional antimicrobials to treat antibiotic resistant bacteria. See, e.g., Kalemba, D.; Kunicka, A. Antibacterial and Antifungal Properties of Essential Oils. Curr. Med. Chem. 2003, 10, 813-829; Hemaiswarya, S.; Kruthiventi, A. K.; Doble, M. Synergism between Natural Products and Antibiotics against Infectious Diseases. Phytomedicine 2008, 15, 639-652. These essential oils and natural compounds are of particular interest as “green” antimicrobial agents due to their low-cost, biocompatibility, and potential anti-biofilm properties. See, e.g., Burt, S. Essential Oils: Their Antibacterial Properties and Potential Applications in Foods—a Review. Int. J. Food Microbiol. 2004, 94, 223-253; Kavanaugh, N. L.; Ribbeck, K. Selected Antimicrobial Essential Oils Eradicate Pseudomonas Spp. and Staphylococcus Aureus Biofilms. Appl. Environ. Microbiol. 2012, 78, 4057-4061; Nostro, A.; Sudano Roccaro, A.; Bisignano, G.; Marino, A.; Cannatelli, M. A; Pizzimenti, F. C.; Cioni, P. L.; Procopio, F.; Blanco, A. R. Effects of Oregano, Carvacrol and Thymol on Staphylococcus Aureus and Staphylococcus Epidermidis Biofilms. J. Med. Microbiol. 2007, 56, 519-523. The generally poor aqueous solubility and stability of these oils has substantially limited their widespread application. See, e.g., Chen, H.; Davidson, P. M.; Zhong, Q. Impacts of Sample Preparation Methods on Solubility and Antilisterial Characteristics of Essential Oil Components in Milk. Appl. Environ. Microbiol. 2014, 80, 907-916. Engineering nanomaterials provides a potential platform to prevent payload degradation and to tune molecular interactions with bacteria. See, e.g., Carpenter, A. W.; Worley, B. V; Slomberg, D. L.; Schoenfisch, M. H. Dual Action Antimicrobials: Nitric Oxide Release from Quaternary Ammonium-Functionalized Silica Nanoparticles. Biomacromolecules 2012, 13, 3334-3342; Zhu, X.; Radovic-Moreno, A. F.; Wu, J.; Langer, R.; Shi, J. Nanomedicine in the Management of Microbial Infection—Overview and Perspectives. Nano Today 2014, 9, 478-498; Radovic-Moreno, A. F.; Lu, T. K.; Puscasu, V. a; Yoon, C. J.; Langer, R.; Farokhzad, O. C. Surface Charge-Switching Polymeric Nanoparticles for Bacterial Cell Wall-Targeted Delivery of Antibiotics. ACS Nano 2012, 6, 4279-4287; Goswami, S.; Thiyagarajan, D.; Das, G.; Ramesh, A. Biocompatible Nanocarrier Fortified with a Dipyridinium-Based Amphiphile for Eradication of Biofilm. ACS Appl. Mater. Interfaces 2014, 6, 16384-16394. Previous reports have shown that encapsulating essential oils into surfactant-stabilized colloidal delivery vehicles improves their aqueous stability and increases the antimicrobial activity of small molecule payloads. See, e.g., Chang, Y.; McLandsborough, L.; McClements, D. J. Physicochemical Properties and Antimicrobial Efficacy of Carvacrol Nanoemulsions Formed by Spontaneous Emulsification. J. Agric. Food Chem. 2013, 61, 8906-8913; Liang, R.; Xu, S.; Shoemaker, C. F.; Li, Y.; Zhong, F.; Huang, Q. Physical and Antimicrobial Properties of Peppermint Oil Nanoemulsions. J. Agric. Food Chem. 2012, 60, 7548-7555; Gomes, C.; Moreira, R. G.; Castell-Perez, E. Poly (DL-Lactide-Co-Glycolide) (PLGA) Nanoparticles with Entrapped Trans-Cinnamaldehyde and Eugenol for Antimicrobial Delivery Applications. J. Food Sci. 2011, 76, N16N24. However, these carriers often induce adverse hemolytic or irritating effects restricting their compatibility with biological tissues. See, e.g., Shalel, S.; Streichman, S.; Marmur, A. The Mechanism of Hemolysis by Surfactants: Effect of Solution Dispersion. J. Colloid Interface Sci. 2002, 252, 66-76; Wilhelm, K.-P.; Freitag, G.; Wolff, H. H. Surfactant-Induced Skin Irritation and Skin Repair. J. Am. Acad. Dermatol. 1994, 30, 944-949.

There remains a continuing need for improved antimicrobial compositions. In particular, crosslinked compositions which utilize highly abundant crosslinking moieties are desirable.

### BRIEF SUMMARY

One embodiment is a crosslinked particle comprising gelatin and an essential oil, wherein at least a portion of the gelatin is crosslinked.

Another embodiment is a composition comprising a plurality of crosslinked particles.

Another embodiment is a method of making the crosslinked particles, the method comprising: combining an essential oil and an aqueous solution comprising gelatin to provide a mixture, wherein at least one of the essential oil or the aqueous solution comprises an initiator; emulsifying the mixture to provide an intermediate composition comprising a plurality of particles comprising a liquid hydrophobic core comprising an essential oil and gelatin; and a shell encapsulating the core, the shell comprising the gelatin; and exposing the intermediate composition to conditions effective to crosslink the gelatin to provide the crosslinked particles.

Another embodiment is a method of treating a bacterial or a fungal infection, the method comprising contacting the above-described composition with a bacterial infection or a fungal infection.

These and other embodiments are described in detail below.

### BRIEF DESCRIPTION OF THE DRAWINGS

The following Figures are of exemplary embodiments.

FIG. 1 is a schematic illustrating the process for making crosslinked particles according to an embodiment.

FIG. 2 shows chemical structures of exemplary components used to prepare the crosslinked particles, including two portions of gelatin (1a, 1b), riboflavin (2), and carvacrol (3).

FIG. 3 is a chemical scheme illustrating an example of gelatin crosslinking through a histidine residue and a hydroxyproline residue, mediated by ultraviolet light and a riboflavin photoinitiator.

FIG. 4 shows an exemplary size distribution by intensity of a sample of the crosslinked particles, obtained using dynamic light scattering, where the particles have an average size of about 340 nanometers.

FIG. 5 shows infrared spectroscopy results that provide insight to the crosslinking mechanism.

FIG. 6 shows the stability of the crosslinked particles after incubation in fetal bovine serum for one hour determined by dynamic light scattering.

FIG. 7 shows images of treated plates and transfer plates containing Fusarium oxysporum fungus without treatment (“control”) and after treatment with the crosslinked particles (“nanocomposite”).

FIG. 8 shows viability of four bacterial biofilms after treatment with increasing amounts of particles (denoted as “GELX Concentration”).

FIG. 9 shows viability of four bacterial biofilms after treatment with varying amounts of particles as well as the individual components that were used to prepare the particles. The individual components do not exhibit significant antimicrobial activity.

### DETAILED DESCRIPTION

The present inventors have discovered crosslinkable compositions stabilized by gelatin and a method for the manufacture thereof. In particular, the present inventors have successfully prepared gelatin-stabilized composites (also referred to herein as gelatin-stabilized sponges, or crosslinked particles), and have found that these composites are stable in aqueous solutions, including serum-containing media, and are further advantageously capable of penetrating bacterial biofilms and eliminating pathogenic bacteria. The composites are robust and the size can conveniently be tuned from nanoscale to microscale to macroscale. The present inventors have also surprisingly found that the composites can be extended to treatment of other infectious diseases such as drug resistant fungi. There is a relatively low number of approved antifungal drugs on the market due to the challenges of treating fungi without long-term adverse environmental effects, and the composites of the present disclosure are expected to have an impact in treating associated fungal infections in agricultural industries. Furthermore, the composites are inherently modular and derived from natural sources, the composites can be extended to applications in food industries.

Accordingly, an aspect of the present disclosure is a crosslinked particle comprising gelatin and an essential oil, wherein at least a portion of the gelatin is crosslinked. In some embodiments, the crosslinked particles include a pervasive crosslinked gelatin network extending through the essential oil to form the composite. In some embodiments, the particles can be considered to have a “core-shell” type morphology where the shell encapsulating the core of the crosslinked particles comprises gelatin, wherein at least a portion of the gelatin is crosslinked. The core comprises the essential oil and an at least partially crosslinked gelatin network In some embodiments, at least 10%, or at least 25%, or at least 40%, or at least 50%, or at least 60%, or at least 75%, or at least 90% of the gelatin is crosslinked. In some embodiments, substantially all of the gelatin is crosslinked.

Gelatin is a product obtained by the partial hydrolysis of collagen derived from the skin, white connective tissue, and bones of animals. It is a derived protein comprising various amino acids linked between adjacent amino and carbonyl groups to provide a peptide bond. The amino acid combinations in gelatin provide amphoteric properties, which are responsible for varying isoelectric values, depending somewhat upon the methods of processing. Important physical properties of gelatin such as solubility, swelling, and viscosity show minimum values at the isoelectric point. In some embodiments, the gelatin can be a recombinant gelatin or a plant-based gelatin. The gelatin can comprise type A gelatin, type B gelatin, or a combination comprising at least one of the foregoing. Type A gelatin results from acid pretreatment (swelling of the raw material in the presence of acid) and is generally made from frozen pork skins treated in dilute acid (HCl, H₂SO₃, H₃PO₄, or H₂SO₄) at a pH of 1 to 2 for 10 to 30 hours, after which it is water washed to remove excess acid, followed by extraction and drying in the conventional manner. Type B gelatin results from alkali pretreatment (swelling of the raw material in the presence of an alkali) and is generally made from ossein or hide stock which is treated in saturated lime water for 3 to 12 weeks, after which the lime is washed out and neutralized with acid. The adjusted stock is then hot water extracted and dried as with type A. Dry bone is cleaned, crushed, and treated for 10 to 14 days with 4 to 7% HCl to remove the minerals (principally tricalcium phosphate) and other impurities before reaching the stage known as ossein. Dry bone is 13 to 17% gelatin whereas dry ossein is 63 to 70% gelatin. Type A gelatin is characterized by an isoelectric zone between pH 7 and 9, whereas type B gelatin has an isoelectric zone between pH 4.7 and 5.0. Thus the ionic character of the gelatin when used as a surfactant can be selected based on the pH of the second solution. Relative to each other, type A gelatin has less color, better clarity, more brittleness in film form and is faster drying than type B. In some embodiments, the gelatin is type B gelatin.

In some embodiments, the gelatin preferably comprises at least one histidine residue and at least one of a hydroxyproline residue, a tyrosine residue, and a threonine residue. The term “residue” as used herein is meant to refer to the portion of the gelatin backbone structure that is derived from the specified amino acid. In some embodiments, the gelatin can include at least one histidine residue and at least one hydroxyproline residue. Without wishing to be bound by theory, it is believed that the presence of these residues can facilitate the crosslinking of the gelatin. It is hypothesized that radical oxidation conditions can convert the imidazole of the histidine residue to an imidazolone, which is susceptible to nucleophilic addition from any of hydroxyproline, histidine, tyrosine, and threonine, preferably hydroxyproline, as it is believed to be the most abundant residue of this group. In some embodiments, the hydroxyproline residue, tyrosine residue, threonine residue, or combination thereof can be present relative to the histidine residue in a molar ratio of at least 2:1, preferably at least 3:1. In some embodiments, the gelatin comprises one or more histidine residues and one or more hydroxyproline residues. For example, the gelatin can have a structure comprising the residues of formula (I) and formula (II)

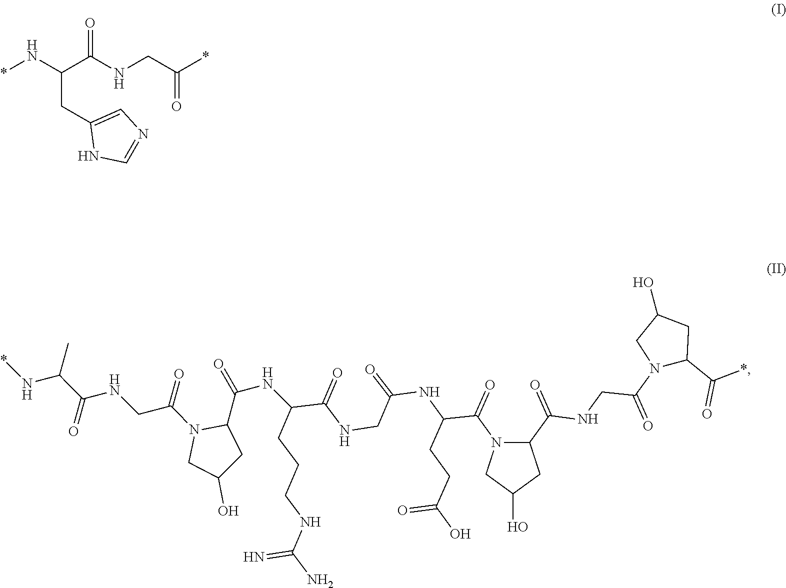

wherein the “*” indicates the point of attachment to of the shown residues to the remainder of the gelatin backbone structure. In some embodiments, the hydroxyproline residues and the histidine residues are present in a molar ratio of at least 2:1, for example at least 3:1.

The gelatin can also comprise one or more lysine residues. Thus, in some embodiments, the gelatin of the particle can optionally be functionalized, for example through reaction at an amine group of a lysine residue. Stated another way, the gelatin can be functionalized at one or more lysine residues of the gelatin. Functionalization can generally be with any moiety containing an amine-reactive group, including, but not limited to, a dye, an imaging agent, a targeting ligand, and the like or a combination thereof. In some embodiments, functionalization can include reacting a fluorescent, luminescent or phosphorescent compound with a pendent amine group on the gelatin, for example reacting fluorescein isothiocyanate or a derivative thereof or tetramethylrhodamine isothiocyanate or a derivative thereof to provide a fluorescein- or rhodamine-labelled particle.

At least a portion of the gelatin in the shell is crosslinked. The gelatin can be crosslinked using an initiator, particularly, in initiator capable of generating singlet oxygen. Photoinitiators, which generate radical species upon irradiation with light, can be particularly useful as initiators. Even more particularly, photoinitiators that produce hydroxyl radicals (.0H) upon irradiation with light can be used. As mentioned above, without wishing to be bound by theory, it is believed that the hydroxyl radicals can oxidize imidazole groups (e.g., of histidine present in the gelatin) to imidazolone groups, which are susceptible to nucleophilic addition from various nucleophilic residues that can be present in gelatin, for example, hydroxyproline, tyrosines, threonines, any remaining (i.e., unoxidized) histidines, and combinations thereof. Any photoinitiator that is generally known to generate hydroxyl radicals can be used. Photoinitiators comprising a flavin group are of particular interest, for example, riboflavin, which can generate hydroxyl radicals upon exposure to ultraviolet radiation (about 370 nanometers).

In some embodiments, the crosslinked gelatin comprises one or more crosslinks of the formula (III)

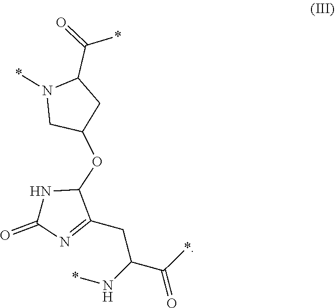

In some embodiments, other polymers can be excluded from the particles described herein.

In addition to the gelatin, the crosslinked particle further comprises an essential oil. The essential oil is a hydrophobic liquid, and is generally a liquid at 25° C. The essential oil can be a naturally occurring compound (e.g., derived from a plant). Essential oils, as used herein, are volatile aromatic oils which can be synthetic or derived from plants (e.g., flowers, buds, seeds, leaves, twigs, bark, herbs, wood, fruits, roots, and the like) by a physical method (e.g., distillation, expression, fermentation, or extraction). Essential oils usually carry the odor or flavor of the plant from which they are obtained. The essential oil is preferably an oil having antimicrobial properties. In some embodiments, the essential oil can be selected from, for example, peppermint oil, oregano oil, thymol, menthol, methyl salicylate, eucalyptol, carvacrol, carvacrol methyl ether, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, lemon oil, orange oil, sage oil, rosemary oil, cinnamon oil, pimento oil, laurel oil, cedar leaf oil, clove oil, cilantro oil, coriander oil, or a combination thereof. In some embodiments, the essential oil is selected from carvacrol oil, limonene, peppermint oil, cilantro oil, coriander oil, cinnamon oil, oregano oil, rosemary oil, sage oil, clove oil, thyme oil, or a combination thereof. In some embodiments, the essential oil comprises carvacrol oil, limonene, or a combination thereof. In some embodiments, the essential oil comprises carvacrol oil.

In some embodiments, the essential oil can have a chemical structure that includes at least one aromatic alcohol group.

In some embodiments, the particle can optionally further include an additive. For example, a hydrophobic antibiotic can be included. The hydrophobic antibiotic is soluble in the essential oil. The presence of the hydrophobic antibiotic can increase the antimicrobial activity of the particle. Suitable hydrophobic antibiotics include nalidixic acid, cinoxacin, norfloxacin, ciprofloxacin, enoxacin, ofloxacin, levofloxacin, sparfloxacin, moxifloxacin, gemifloxacin, trovafloxacin, ampicillin, amoxicillin, carbenicillin, carfecillin, ticarcillin, azlocillin, mezlocillin, piperacillin, cefepime, tetracycline, gentamicin, tobramycin, streptomycin, neomycin, kanamycin, amikacin, cefoselis, and cefquinome. In some embodiments, the additive can be any compound having antimicrobial activity, including, for example, a cycloheptatriene or derivative thereof, β-Thujaplicin, tropolone, tropone, stipitatic acid, puberulic acid, puberulonic acid, or a combination thereof. When present, the additive can be included in the particle in an amount of 1 to 10 weight percent, based on the weight of the essential oil.

As described above, in some embodiments, particles can be considered to hava a core-shell structure. In this embodiment, the “core” of the particle comprises the essential oil and gelatin, which can be crosslinked or uncrosslinked. In some embodiments, a majority of the gelatin included in the particle is present at the periphery of the particle (i.e., forming a shell). For example, a shell can include at least 50 weight percent of the gelatin (based on the total weight of the gelatin in the particle), or at least 60 weight percent, or at least 75 weight percent, or at least 85 weight percent, or at least 95 weight percent, or at least 99 weight percent.

The crosslinked particles can be of a range of sizes depending on the desired application. For example, in some embodiments, the particles can be nanoparticles having an average diameter of less than or equal to 500 nanometers (nm), for example 1 to 500 nm, or 10 to 500 nm, or 50 to 500 nm, or 75 to 450 nm, or 100 to 450 nm, or 150 to 450 nm, or 200 to 450 nm, or 250 to 450 nm, or 275 to 400 nm, or 300 to 400 nm. The average diameter of the particle can be determined, for example, using light scattering techniques.

In some embodiments, the crosslinked particle comprises 90 to 99.9 weight percent, or 90 to 99 weight percent, or 95 to 99 weight percent of the essential oil and 0.1 to 10 weight percent, or 1 to 10 weight percent, or 1 to 5 weight percent of the gelatin, wherein weight percent is based on the total weight of the particle.

In an embodiment, the crosslinked particle comprises, based on the total weight of the particle, 1 to 10 weight percent of the gelatin and 90 to 99 weight percent of the essential oil. The essential oil can comprise carvacrol oil, and the gelatin can be substantially crosslinked, comprising one or more crosslinks according to formula (III). Preferably, no other polymers are present in the particle.

Another aspect of the present disclosure is a composition comprising a plurality of the crosslinked particles. As used herein, “a plurality of particles” refers to a composition comprising more than 1 particle, for example more than 10 particles. The composition can also be referred to as a dispersion. In some embodiments, the composition comprises 0.01 to 90 weight percent of the particles. The particles can have the above-described structure and components. In some embodiments, the composition can be in the form of a gel, a cream, or a paste (e.g., a toothpaste, a topical ointment, and the like). In some embodiments, the composition comprises 50 to 90 weight percent, or 50 to 80 weight percent of the particles, based on the total weight of the composition. In some embodiments, the particles are dispersed in a liquid carrier, for example an aqueous solution or a C₁₋₆ alcohol (e.g., ethanol), preferably an aqueous solution. Thus, in some embodiments, the composition can be an aqueous composition, in which the crosslinked particles are dispersed in an aqueous solution. In such an embodiment, the composition is in the form of a liquid. In some embodiments, the composition comprises 0.01 to 50 weight percent of the particles, based on the total weight of the composition. The aqueous solution can comprise water, deionized water, a buffer (e.g., phosphate buffered saline, phosphate buffer, and the like), and the like, or a combination thereof. The composition can optionally further comprise various additives that are generally known in the art, with the proviso that the additives do not significantly adversely affect one or more desired properties of the composition. Furthermore, it can be particularly desirable that the presence of any additive does not significantly interfere with the structure of the particles. Additives can include stabilizers, thickeners, viscosity enhancers, coloring agent, surfactants, emulsifiers, humectants, and the like, or a combination thereof.

Another aspect of the present disclosure is a method of making the crosslinked particles. The method comprises combining an essential oil and an aqueous solution to provide a mixture. The aqueous solution comprises the gelatin, and at least one of the essential oil or the aqueous solution comprises an initiator. The initiator can be capable of generating singlet oxygen to effect crosslinking of the gelatin. The method further comprises emulsifying the mixture to provide an intermediate composition. Emulsification can be accomplished by any known method including, but not limited to shaking (e.g., hand-shaking), stirring, or by using an amalgamator, or any other suitable mixing device. Emulsifying the mixture provides a plurality of particles comprising the essential oil and the crosslinked gelatin. Without wishing to be bound by theory, the gelatin can stabilize the oil/water interface of the particles in aqueous solution. The intermediate particles can then be exposed to conditions effective to crosslink the gelatin to provide the crosslinked particles. In some embodiments, particularly when a photoinitiator is used, the conditions effective to crosslink the gelatin can comprise exposing the intermediate composition to ultraviolet (UV) irradiation. Exposure to the UV irradiation can be for a time of 1 to 30 minutes, or 5 to 20 minutes, and at a temperature of 10 to 30° C., or 15 to 25° C., or 20 to 25° C.

Another aspect of the present disclosure is a method of treating a bacterial or fungal infection. The method comprises contacting the above-described composition comprising the crosslinked particles with a bacterial or fungal infection. In some embodiments, the composition comprising the particles can be used as an injectable antimicrobial or antifungal formulation and can treat a bacterial infection or fungal infection in vivo. In some embodiments, the composition comprising the particles can be used as a topical treatment for a bacterial or fungal infection.

In some embodiments, the crosslinked particles can be used in a method of treating a bacterial infection or a bacterial biofilm. A “biofilm” refers to a population of bacteria attached to an inert or living surface. Thus, biofilms can form on a counter, a table, water pipes, implants, catheters, cardiac pacemakers, prosthetic joints, cerebrospinal fluid shunts, endotracheal tubes, and the like. In some embodiments, the biofilm can be present on a living surface, for example skin or in a wound, and on teeth (e.g., dental plaque). Bacteria in a biofilm are enmeshed in an extracellular polymer matrix, generally a polysaccharide matrix, which holds the bacteria together in a mass, and firmly attaches the bacterial mass to the underlying surface. Evidence has shown that biofilms constitute a significant threat to human health. Wounds and skin lesions are especially susceptible to bacterial infection.

In some embodiments, the bacterial biofilm can be a gram-negative bacterial biofilm or a gram-positive bacterial biofilm. In some embodiments, the bacterial biofilm comprises Escherichia coli (e.g., E. coli DH5α), Pseudomonas bacteria (e.g., Pseudomonas aeruginosa), Staphylococcal bacteria (e.g., Staphylococcal aureus), Enterobacteriaceae bacteria (e.g., E. cloacae complex), Streptococcus bacteria, Haemophilus influenzae, Leptospira interrogans, Legionella bacteria, Mycobacterium tuberculosis, Candida albicans bacteria, Acinetobacter baumannii bacteria, Stenotrophomonas maltophilia bacteria, Clostridium difficile bacteria, Enterococcus bacteria, Klebsiella pneumoniae bacteria, Necrotizing fascitis bacteria, Corynebacterium bacteria, Helicobacter pylori bacteria, Campylobacter bacteria, Salmonellae bacteria, Neisseria gonorrhoeae bacteria, Haemophilus influenza bacteria, Shigella bacteria, or a combination thereof.

Contacting the composition comprising the particles with a biofilm can effectively kill bacterial cells present in the biofilm. Accordingly, the compositions disclosed herein can be particularly useful as disinfectants or antimicrobial compositions. The contacting can be under conditions effective to treat the biofilm, for example for a time of 10 minutes to 5 hours, or 1 hour to 3 hours, and at a temperature of 25 to 37° C. As used herein, “treating a biofilm” can refer to killing at least 20%, or at least 40%, or at least 50%, or at least 60%, or at least 80%, or at least 90% of the bacterial cells present in the biofilm. In some embodiments, contacting the composition with a biofilm can completely remove the biofilm (i.e., the dispersion is toxic to greater than 90%, or 99% or 99.9% of the bacterial cells of the biofilm upon contacting the dispersion with the biofilm).

In another embodiment, the crosslinked particles can be used in a method of treating a fungal infection. The fungal infection can comprise, for example, Alternaria alternata, Aspergillus fumigatus, Aspergillus niger, Aspergillus flavus, Aspergillus nidulans, Aspergillus paraciticus, Candida albicans, Candida dubliniensis, Candida famata, Candida glabrata, Candida guilliermondii, Candida haemulonii, Candida kejyr, Candida Candida lusitaniae, Candida norvegensis, Candida parapsilosis, Candida tropicalis, Candida viswanathii, Epidermophyton floccosurn, Fusarium grarninearurn, Fusarium oxysporum (e.g., Fusarium oxysporum f.sp. lycopersici, Fusarium oxysporum f.sp. cubense, Fusarium oxysporum f.sp. lini, Fusarium oxysporurn f.sp. vasinfectum, Fusarium oxysporum f.sp. batatas, and Fusarium oxysporum f.sp. nicotianae), Fusarium solani, Fusarium monoliforme, Tryehophyton rubrum, Trychophyton mentagrophytes, Trychophyton inter digitales, Trychophyton tonsurans, Cryptococcus neoformans, Cryptococcus gattii, Cryptococcus grubii. Colletotriehurn graminicola, Microsporum canis, Microsporurn gypseum, Penicillium marneffei, Tricosporon beigelii, Trichosporon asahii, Trichosporon inkin, Trichosporon asteroides, Trichosporon cutaneum, Trichosporon domestieum, Trichosporon mucoides, Trichosporon ovoides, Trichosporon pullulans, Triehosporon loubieri, Trichosporon japonicum, Seedosporium apiospermum, Scedosporium prolifwans, Paecilomyces variotii, Paecilomyces lilacinus, Acremonium strieutin, Cladophialophora bantiana, Wangiella dermatitidis, Ramichioridium obovoideum, Chaetomium atrobrunneum, Dactiaria gallopavum, Bipolaris spp, Exserohilum rostratum, Absidia comnbifera, Apophysomyces elegans, Mucor indicus, Rhizomucor pusillus, Rhizopus oryzae, Cunninghamelia bertholletiae, Cokeromyces recurvatus, Saksenaea vasitbrmis, Syncephaiastrum racemosum, Basidiobolus ranarum; Conidiobolus coronatuslConidiobolus incongruus, Blastomyces dermatitidis, Coccidioides immitis, Coccidioides posadasii, Histoplasma capsulatum, Paracoccidioides brasitiensis, Pseudallescheria boydii, Sporothrix schenckii, Alternaria brassicicola, Alternaria alternata, Aspergillus nidulans, Botrytis cinerea, Cercospora beticola, Cercospora zeae maydis, Cochliobohis heterostrophus, hxserohilum turcicum, Fusarium culmorum, Fusarium oxysporum, Fusarium oxysporum f. sp. dianthi, Fusarium solani, fusarium pseudogramineanim, Fusarium verticilioides, Gaeumannomyces graminis var. tritici; Plasmodiophora brassicae, Scierotinia scierotiorum, Stenocarpelia (Diplodia) maydis, Thielaviopsis basicola, Verticillium dahliae, Ustilago zeae, Puccinia sorghi, Macrophomina phaseolina, Phialophora gregata, Diaporthe phaseolorum, Cercospora Phytophthora sojae, Rhizoctonia solani, Phakopsora pachyrhizi, Alternaria macrospore, Cercospora gossypina, exigua, Puccinia schedonnardii, Puccinia cacabata, Phymatotrichopsis omnivora, Fusarium avenaceum, Alternaria brassicae, Alternaria raphani, Erysiphe graminis (Blumeria graminis), Septoria tritici, Septoria nodorum, Mycosphaerella zeae, Rhizoctonia cerealis, Ustilago tritici, Puccinia graminis, Puccinia triticina, Tilletia indica, Tilletia caries, Tilletia controversa, Alternaria solani, Alternaria brassicae, Aiternaria brassicoia, Monolinia fructicola, Venturia inaequalis, Ciadosporum carpophilum, Botryosphaeria obtuse, Monilinia vaccinia-corymbosi, Scierotinia homoeocarpa, Podosphaera xanthii, Podosphaera Erysiphe cichoracearum, Blumeria graminis f. sp. Tritici, blumeria graminis f. sp. Hordei, Microsphaera diffusa, Erysiphe necator, Leveilluia Taurica, Podosphaera leucotricha, Podosphaera aphanis, Sawadaea tulasnei, Erysiphe berberidis, Golovinomyces orontii, Peronospora beibahrii, Pseudoperonospora cubensis, Plasmopara viticola, Pseudoperonospora humuti, Peronospora manshurica, Plasinopara halstedii, Phytopthora capcisi, Phytopthora infestans, Phytopthora cinnamomic, Phytopthora sojae, Phytopthora agathidicida, Phytopthora cactorum, Phytopthora citricoia, Phytopthora fragariae, Phytopthora kernoviae, Phytopthora lateralis, Phytopthora megakarya, Phytopthora muitivora, Phytopthora nicotianae, Phytopthora pahnivora, Phytopthora ramorum, Phytopthora quercina, or a combination thereof. In some embodiments, the fungal infection comprises Fusarium oxysporum.

Contacting the composition comprising the particles with a fungus can effectively kill fungal cells present. Accordingly, the compositions disclosed herein can be particularly useful as an antifungal composition. The contacting can be under conditions effective to treat the fungus, for example for a time of 10 minutes to 5 hours, or 1 hour to 3 hours, and at a temperature of 25 to 37° C. As used herein, “treating a fungus” can refer to killing at least 20%, or at least 40%, or at least 50%, or at least 60%, or at least 80%, or at least 90% of the fungal cells present. In some embodiments, contacting the composition with a fungus can completely remove the fungus (i.e., the composition is toxic to greater than 90%, or 99% or 99.9% of the fungal cells of a fungal growth film upon contacting the composition with the fungus).

In summary, the present disclosure provides crosslinked, gelatin-stabilized particles comprising an essential oil. The crosslinked particles demonstrate highly effective therapeutic behavior, successfully eradicating pathogenic biofilm strains of clinical isolates as well as drug-resistant fungal infections. These particles have potential applications as a general surface disinfectant, an antiseptic for wound treatment, and as an antifungal. The self-assembly and crosslinking method used to prepare the particles provides a promising platform to create effective delivery vehicles to combat bacterial and fungal infections.

The invention is further illustrated by the following non-limiting examples.

### Examples

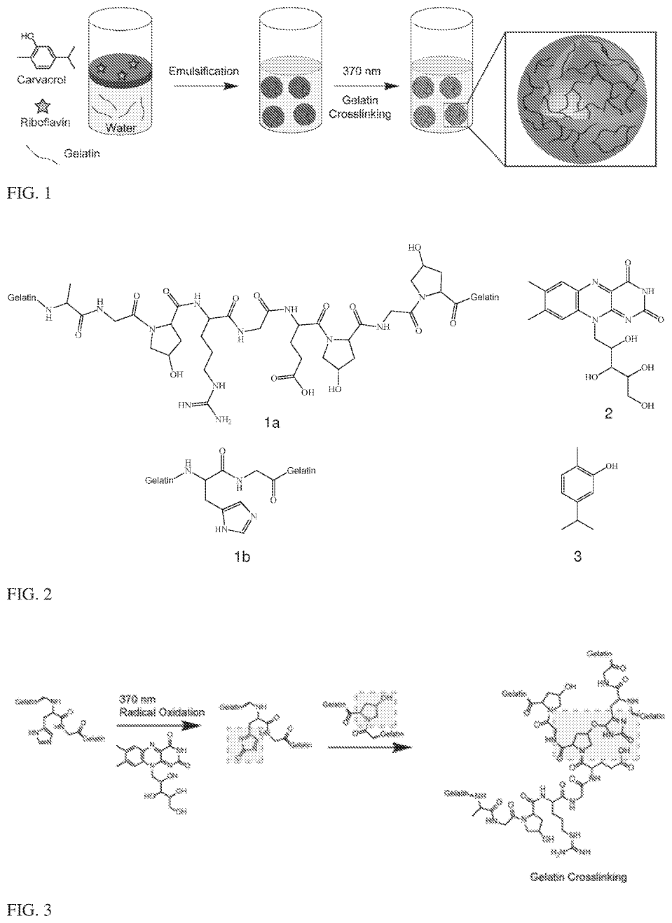

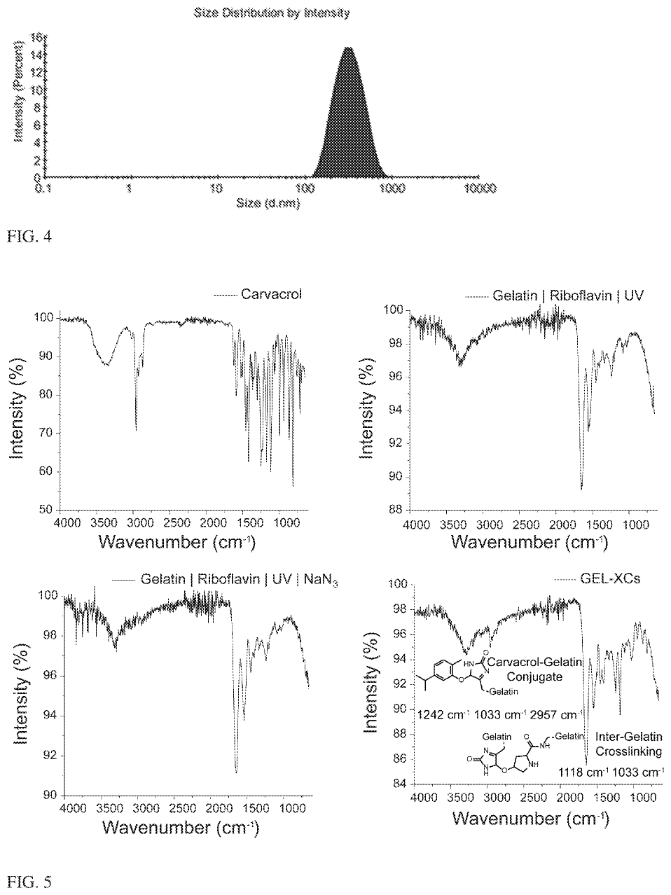

The particles were prepared according to the general scheme shown in FIG. 1. The chemical structures of each component are shown in FIG. 2. Specifically, a typical crosslinked particle was prepared by adding riboflavin (3 milligrams) to carvacrol oil (3.07 milliliters, available from Sigma, CAS Reg. No. 499-75-2). Gelatin (type B, molecular weight=20,000 g/mol, available from Fisher Scientific) was dissolved in water to provide an aqueous solution having a gelatin concentration of 3 milligrams per milliliter (total volume, 40 milliliters). The aqueous solution of gelatin (40 milliliters) was added to the riboflavin/carvacrol oil mixture. The mixture was homogenized using an IKA T25 homogenizer for 99 seconds as a speed of 25,000 RPM. The resulting particles were then irradiated with UV light at 365 nanometers while stirring for 20 minutes at about 25 degrees Celsius to provide the crosslinked particles. The proposed mechanism of crosslinking is shown in the schematic illustration of FIG. 3.

After irradiation, the emulsion solution became more apparently viscous and opaque. Notably, emulsion controls including the absence of either gelatin, riboflavin, or UV light did not generate stable emulsions and fell apart one day after emulsification (in the case of no gelatin present, no emulsion was observed).

The crosslinked gelatin particles were characterized using dynamic light scattering to determine particle size and dispersity. FIG. 4 shows DLS results of a typical sample prepared according to the above procedure, where the resulting crosslinked particles have an average diameter of 300 nanometers.

Notably, crosslinked gelatin particles demonstrated high shelf-life, maintaining its inherent size even after on the benchtop for 2 years.

Morphology of the crosslinked gelatin particles was observed by confocal microscopy by generating micrometer sized emulsion analogs. First, gelatin was labelled with a blue fluorescent coumarin dye through its residual lysine residues that are believed to take no part in the crosslinking process. Next, green fluorescent DiO dye was loaded within the oil to clearly juxtapose the oil core from the gelatin stabilizer. Micrometer-sized emulsions were viewed under confocal and analyzed with Image J software. The results indicated that gelatin can be observed to co-localized with the oil core in addition to localizing at the oil-water interface and beyond. Without wishing to be bound by theory, it is believed that gelatin's native structure contains a large number of proline and hydroxyproline residues, imparting β-sheet structures. Additionally, gelatin contains numerous hydrophobic residues that carvacrol oil can embed within. Crosslinked gelatin would in theory be a more hydrophobic material given the number of hydrophobic residues outweigh its polar residue domains. Explanation of gelatin present beyond the oil can be explained during its formulation process. Although riboflavin has some solubility in carvacrol, it is not completely soluble. Therefore, during UV irradiation, riboflavin can leak into the water and enable crosslinking units beyond the oil. Residual gelatin outside of the emulsions can attach later on. This is further supported in the confocal as entire emulsions can be crosslinked and are linked through these exterior gelatin appendages. No evidence of inter-emulsion crosslinking can be observed under DLS when nano-sized emulsions are formed, suggesting that this type of crosslinking occurs during emulsification of a larger volume oil. Taken together, crosslinked gelatin particles largely adopt a composite morphology with hydrogel-like appendages.

The chemical properties of the crosslinked gelatin particles were also explored, particularly the crosslinking mechanism. As shown in FIG. 5, attenuated total reflectance Fourier transform infrared spectroscopy (ATR-FTIR) was used to monitor inter-gelatin crosslinking and carvacrol-gelatin crosslinking. FIG. 5 shows the ATR spectra for carvacrol (top left) with its indicative peaks, including a phenol stretch at 3337 cm⁻¹, aliphatic C-H stretches at 2959, 2927, and 2860 cm⁻¹, alkane bending at 1457 and 1419 cm⁻¹, and C-O stretch at 1250 cm⁻¹. Next, three individual reactions were setup to monitor changes in gelatin's IR frequencies. The first reaction (top right, positive control) mixes riboflavin and gelatin in the presence of UVA light for 30 minutes, the second (bottom left, negative control) performs the same reaction however in the presence of sodium azide, a well-known quencher of singlet oxygen. The final experiment (bottom right) is generation of crosslinked gelatin particles. All three reactions underwent dialysis and were lyophilized to remove by-product noise (riboflavin, residual carvacrol oil, sodium azide, and water). The results indicate that inter-gelatin crosslinking occurs (aliphatic ether formation: 1118 cm⁻¹, aliphatic-aromatic ether formation: 1033 cm⁻¹) in the positive control, however these signatures are completely absent in the negative control. Interestingly, IR spectra of the crosslinked gelatin particles not only shows inter-gelatin crosslinking signatures (stronger in frequency due to gelatin concentration at the oil-water interface), but signatures from gelatin-carvacrol conjugation can also be seen (broadening of 1033 cm⁻¹ and additional aromatic ether signature at 1242 cm⁻¹). Furthermore, obvious carvacrol conjugation can be seen due to appearance of sp3 C-H stretches at 2957 cm⁻¹ and is nearly identical to the carvacrol oil IR. Taken together with the observations from confocal microscopy, carvacrol serves to not only enable inter-gelatin crosslinking through oil-templating, but conjugates onto gelatin, imparting more hydrophobicity and allow gelatin to better transverse the oil core.

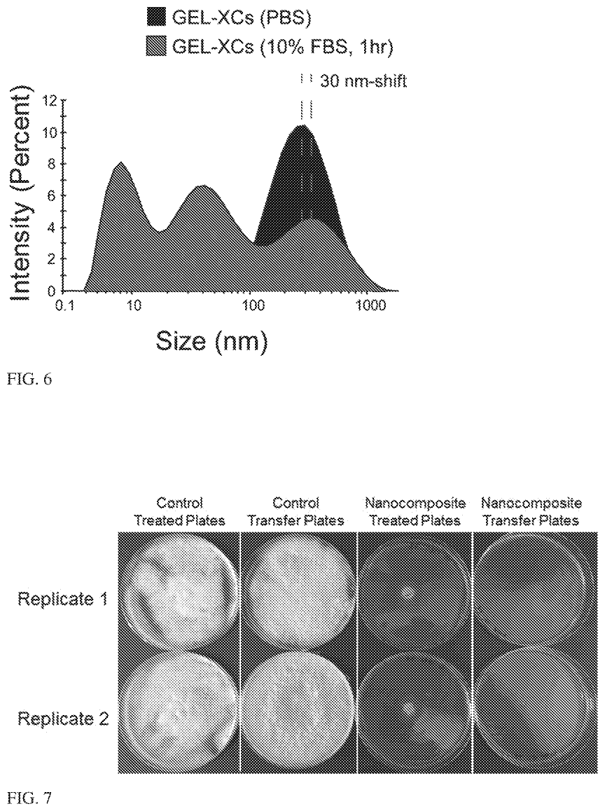

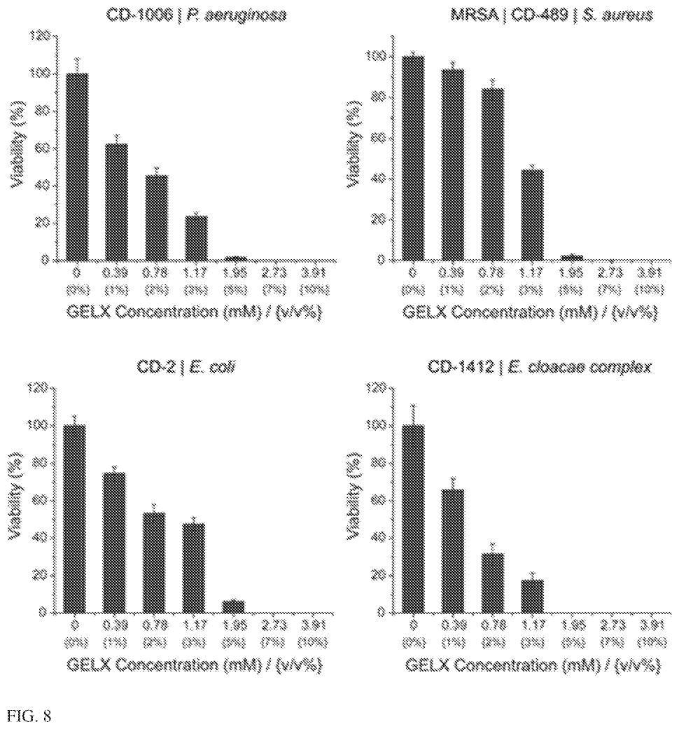

Stability of the crosslinked gelatin particles was also evaluated in a complex biological environment. The stability of the crosslinked gelatin particles was evaluated in 10% fetal bovine serum (FBS) as shown in FIG. 6, using dynamic light scattering (DLS) and compared to the size determined in 150 mM phosphate buffered saline (PBS). After incubating the crosslinked gelatin particles in FBS for 1 hour, a size increase of 30 nanometers was observed, likely due to serum protein adsorption. The presence of carvacrol can induce protein unfolding of BSA, and this process is thought to occur on the surface of the crosslinked gelatin particles.

### Treatment of a Fungal Infection

Spores of F. oxysporum 4287 were inoculated into Potato Dextrose Broth (PDB) and shaken for 3 days at 28° C. An unfiltered culture sample was vortexed and spotted into the center of PDA plates and left to grow for 2 days at 28° C. Colonies were covered in either water (referred to as “control”) or nanoparticles ensuring complete covering of the colony. Plates were placed back into 28° C. and left to grow for 2 days. A scraping of each colony was taken from the treated plate and transferred onto fresh PDB. Plates were placed at 28° C. and left to grow for 7 days. Colony diameter was measured prior to treatment.

As shown in FIG. 7, water treated plates and water transferred plates show significant fungal growth. In contrast, nanoparticle (i.e., “nanocomposite”) treated plates showed no growth post-treatment and nanocomposite transfer plates similarly showed no growth.

### Treatment of a Bacterial Biofilm

Bacteria were cultured in LB broth medium obtained from Fisher BioReagents at 37° C. The bacterial cultures were then harvested by centrifugation and washed with 0.85 wt. % aqueous sodium chloride solution three times. Concentrations of resuspended bacterial solution were determined by optical density measured at 600 nanometers. Seeding solutions were then made in minimal M9 broth obtained from Teknova to reach an optical density of 0.1 determined at 600 nanometers. 100 μL of the seeding solutions were added to each well of the 96-well plate. M9 medium without bacteria was used as a negative control. The plates were then covered and incubated at room temperature under static conditions for a desired period. Planktonic bacteria were removed by washing with phosphate buffered saline (PBS) three times.

Solution of crosslinked particles of varying concentrations (0, 1, 2, 3, 5, 7, and 10 vol. %) were made in MEM, and incubated with the washed biofilms at 37° C. in the absence of light. Biofilms incubated with a solution having 0 vol. % of the crosslinked particles was used as a growth control. After three hours, biofilms were washed with phosphate buffered saline (PBS) three time, and the viabilities were determined using an Alamar Blue assay according to the manufacturer's protocol. (Invitrogen Biosource). As shown in FIG. 8, the crosslinked particles exhibit antimicrobial behavior. Even for a 1 vol. % solution for P. aeruginosa, viability was dramatically reduced to only about 60%, and to about 75% for E. coli and 65% for E. cloacae complex. No marked decrease in viability was noted for S. aureus bacteria until the concentration of particles was increased to 3 vol. %, where viability reduced to about 45%. In all cases, for compositions having 5 vol. % particles, viability was reduced to about 10% or less, and compositions having 7 vol. % or greater exhibited no viability. Thus, the crosslinked particles have been shown to effectively treat various bacterial biofilms.

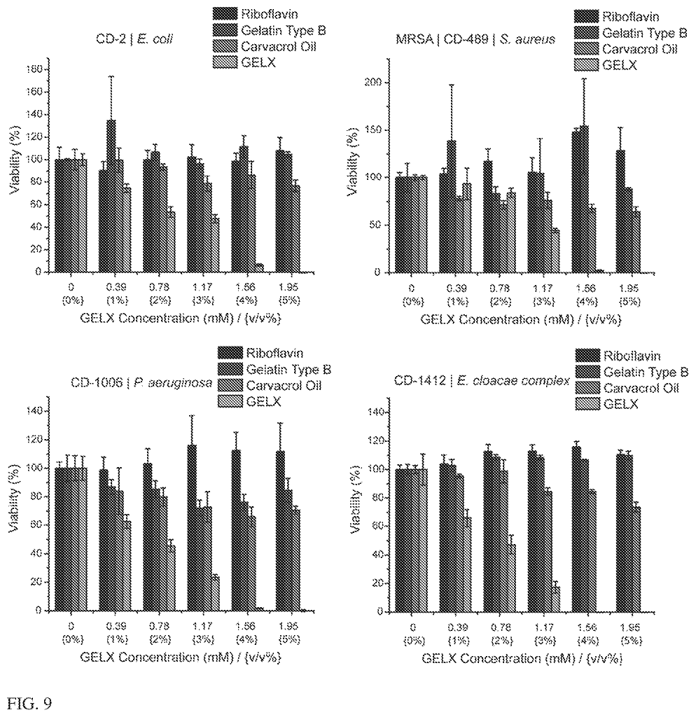

The same experimental conditions were performed for each individual component of the crosslinked particles as well to ensure that any observed antimicrobial activity for the particles was due to the crosslinked particles and not an individual component, such as riboflavin, which is a known antimicrobial agent that generates reactive oxygen species when irradiate with light. As shown in FIG. 9, the individual components did not exhibit significant antimicrobial activity, and in particular did not exhibit antimicrobial activity to the same degree observed for the particles.

The invention includes at least the following embodiments.

### Embodiment 1

A crosslinked particle comprising gelatin and an essential oil, wherein at least a portion of the gelatin is crosslinked.

### Embodiment 2

The crosslinked particle of embodiment 1, wherein the particle is a nanoparticle, preferably wherein the nanoparticle has an average diameter of 1 to 500 nanometers.

### Embodiment 3

The crosslinked particle of embodiment 1 or 2, wherein the essential oil is selected from the group consisting of peppermint oil, oregano oil, thymol, menthol, methyl salicylate, eucalyptol, carvacrol, carvacrol methyl ether, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, lemon oil, orange oil, sage oil, rosemary oil, cinnamon oil, pimento oil, laurel oil, cedar leaf oil, clove oil, cilantro oil, coriander oil, or a combination thereof, preferably wherein the oil comprises carvacrol oil, limonene, peppermint oil, cilantro oil, coriander oil, cinnamon oil, oregano oil, rosemary oil, sage oil, clove oil, thyme oil, or a combination thereof, more preferably wherein the oil comprises carvacrol.

### Embodiment 4

The crosslinked particle of any one of embodiments 1 to 3, wherein the gelatin comprises a histidine residue and at least one of a hydroxyproline residue, a tyrosine residue, and a threonine residue.

### Embodiment 5

The crosslinked particle of embodiment 4, wherein the gelatin comprises a histidine residue and a hydroxyproline residue, wherein the hydroxyproline residue and the histidine residue are present in a molar ratio of at least 2:1, preferably at least 3:1.

### Embodiment 6

The crosslinked particle of any one of embodiments 1 to 5, wherein the gelatin has a structure comprising the residues of formula (I) and formula (II)

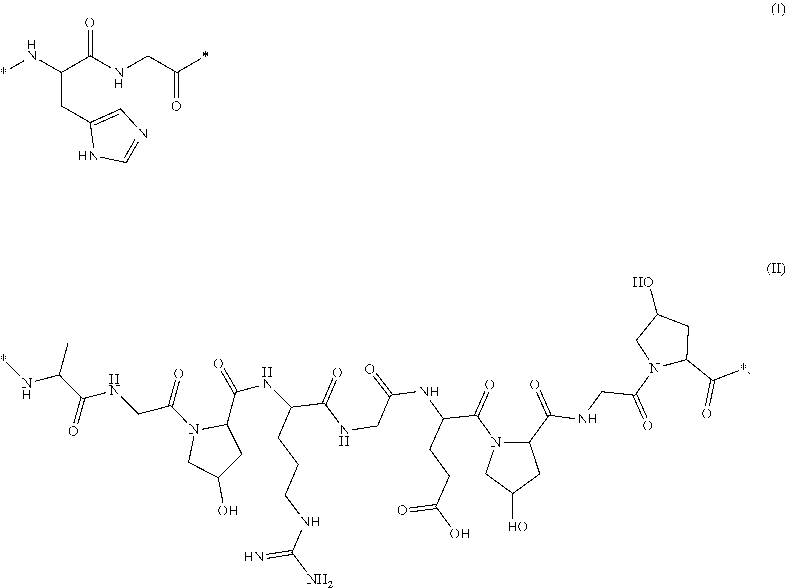

### Embodiment 7

The crosslinked particle of any of embodiments 1 to 6, wherein the gelatin comprises Type B gelatin, Type A gelatin, or a combination thereof

### Embodiment 8

The crosslinked particle of any of embodiments 1 to 7, wherein the particle has a core-shell structure, wherein the core of the particle comprises the essential oil and gelatin that is at least partially crosslinked, and the shell comprises crosslinked gelatin.

### Embodiment 9

The crosslinked particle of any of embodiments 1 to 8, wherein the gelatin is crosslinked using a photoinitiator, preferably wherein the photoinitiator comprises a flavin group, more preferably wherein the photoinitiator comprises riboflavin.

### Embodiment 10

The crosslinked particle of any of embodiments 1 to 9, wherein the crosslinked gelatin comprises one or more crosslinks of formula (III)

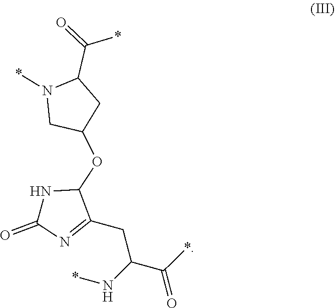

### Embodiment 11

The crosslinked particle of any of embodiments 1 to 10, comprising: 90 to 99.9 weight percent of the essential oil; and 0.1 to 10 weight percent of the gelatin; wherein weight percent is based on the total weight of the particle.

### Embodiment 12

A composition comprising a plurality of crosslinked particle according to any of embodiments 1 to 11, preferably wherein the crosslinked particle are dispersed in an aqueous solution.

### Embodiment 13

A method of making the crosslinked particles of any of embodiments 1 to 11, the method comprising: combining an essential oil and an aqueous solution comprising gelatin to provide a mixture, wherein at least one of the essential oil or the aqueous solution comprises an initiator; emulsifying the mixture to provide an intermediate composition comprising a plurality of particles comprising a liquid hydrophobic core comprising an essential oil and gelatin; and a shell encapsulating the core, the shell comprising the gelatin; and exposing the intermediate composition to conditions effective to crosslink the gelatin to provide the crosslinked particles.

### Embodiment 14

The method of embodiment 13, wherein the conditions effective to crosslink the gelatin comprise exposing the intermediate composition to ultraviolet irradiation.

### Embodiment 15

The method of embodiment 13 or 14, wherein the initiator comprises a photoinitiator, preferably wherein the photoinitiator comprises a flavin group, more preferably wherein the photoinitiator comprises riboflavin.

### Embodiment 16

A method of treating a bacterial or a fungal infection, the method comprising contacting the composition of embodiment 12 with a bacterial infection or a fungal infection.

### Embodiment 17

The method of embodiment 16, comprising contacting the composition of claim 12 with a bacterial biofilm, wherein the bacterial infection comprises Escherichia coli, Pseudomonas bacteria, Staphylococcal bacteria, Enterobacteriaceae bacteria, Streptococcus bacteria, Haemophilus influenzae, Leptospira interrogans, Legionella bacteria, Mycobacterium tuberculosis, Candida albicans bacteria, Acinetobacter baumannii bacteria, Stenotrophomonas maltophilia bacteria, Clostridium difficile bacteria, Enterococcus bacteria, Klebsiella pneumoniae bacteria, Necrotizing fascitis bacteria, Corynebacterium bacteria, Helicobacter pylori bacteria, Campylobacter bacteria, Salmonellae bacteria, Neisseria gonorrhoeae bacteria, Haemophilus influenza bacteria, Shigella bacteria, or a combination thereof.

### Embodiment 18

The method of embodiment 16, comprising contacting the composition of embodiment 12 with a fungal infection, wherein the fungal infection comprises Alternaria alternata, Aspergillus furnigatus, Aspergillus niger, Asperigillus flavus, Aspergillus nidulans, Aspergillus paraeitieus, Candida albicans, Candida Candida farnata, Candida glabrata, Candida guilliermondii, Candida haemulonii, Candida kejyr, Candida krusei, Candida lusitaniae, Candida norvegensis, Candida parapsilosis, Candida tropicalis, Candida viswanathii, Epidermophyton floccosurn, Fusarium graminearum, Fusarium oxysporum, Fusarium solani, Fusarium monoliforme, Trychophyton rubrum, Trychophyton mentagrophytes, Trychophyton inter digitales, Trychophyton tonsurans, Cryptococcus neoformans, Cryptococcus gattii, Cryptococcus grubii, Coiletotrichum graminicola, Microsporum canis, Microsporum gypseum, Penicillium mameffei, Tricosporon beigelii, Trichosporon asahii, Trichosporon inkin, Trichosporon asteroides, Trichosporon cutaneum, Trichosporon domesticum, Trichosporon mucoides, Trichosporon ovoides, Trichosporon pullulans, Trichosporon loubieri, Trichosporon japonicum, Scedosporium apiospermum, Scedosporium prolifwans, Paecilomyces variotii, Paecilomyces lilacinus, Acremonium stricutm, Cladophialophora bantiana, Wangiella dermatitidis, Ramichloridium obovoideum, Chaetomium atrobrunneum, Dactiaria gallopavurn, Bipolaris spp, Exserohilum rostratum, Absidia corymbifera, Apophysomyces elegans, Mucor indicus, Rhizornucor pusillus, Rhizopus oryzae, Cunninghamella berthoilletiae, Cokeromyces recurvatus, Saksenaea vasiformis, Syncephalastrum racemosum, Basidiobolus ranarum, Conidiobolus coronatusl Conidiobolus incongruus, Blastornyces dermatitidis, Coccidioides immitis, Coccidioides posadasii, Histoplasma capsulaturn, Paracoccidioides brasiliensis, Pseudaliescheria boydii, Sporothrix schenckii, Alternaria brassicicola, Alternaria alternata, Aspergillus nidulans, Botrytis cinerea, Cercospora beticola, Cercospora zeae maydis, Cochliobolus heterostrophus, Exserohilum turcicum, Fusarium cuhnorum, Fusarium oxysporum, Fusarium oxysporum f. sp. dianthi, Fusarium solani, Fusarium pseudograminearum, Fusarium verticifloides, Gaeumarmomyces graminis var. tritici, Plasmodiophora brassicae, Scierotinia sclerotiorum, Stenocarpella (Diplodia) maydis, Thielaviopsis basicola, Verticillium dahliae, Ustilago zeae, Puccinia sorghi, Macrophomina phaseolina, Phialophora gregata, Diaporthe phaseolorum, Cercospora sojina, Phytophthora sojae, Rhizoctonia solani, Phakopsora pachyrhizi, Alternaria macrospora, Cercospora gossypina, Piaorria exigua, Puccinia schedonnardii, Puccinia cacabata, Phymatotrichopsis omnivora, Fusarium avenaceum, Alternaria brassicae, Alternaria raphani, Erysiphe graminis (Blumeria graminis), Septoria tritici, Septoria nodorum, Mycospiaaereila zeae, Rhizoctonia cerealis, Ustilago tritici, Puccinia graminis, Puccinia triticina, Tilletia indica, Tilletia caries, controversa, Alternaria solani, Alternaria brassicae, Alternaria brassicola, Monolinia fructicola, Venturia inaequalis, Cladosporum carpophilurn, Botryosphaeria obtuse, Monilinia vaccinia-corymbosi, Sclerotinia homoeocarpa, Podosphaera xanthii, Podosphaera fuliginea, Erysiphe cichoracearum, Blumeria graminis f. sp. Tritici, biumeria graminis f. sp. Hordei, Microsphaera cliffusa, Erysiphe necator, Leveillula Taurica, Podosphaera leucotricha, Podosphaera aphanis, Sawadaca tulasnei, Erysiphe berberidis, Golovinornyces orontii, Peronospora belbahrii, Pseudoperonospora cubensis, Plastnopara viticola, Pseudoperonospora humuii, Peronospora manshurica, Piasmopara halstedii, Phytopthora capcisi, Phytopthora infestans, Phytopthora cinnarnornic, Phytopthora sojae, Phytopthora agathidicida, Phytopthora cactorum, Phytopthora citricola, Phytopthora fragariae, Phytopthora kernoviae, Phytopthora lateralis, Phytopthora megakarya, Phytopthora multivora, Phytopthora nicotianae, Phytopthora palmivora, Phytopthora rarnorum, Phytopthora quercina, or a combination thereof.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety, including U.S. priority application No. 62/598,070, filed Dec. 13, 2017. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range. The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

1. A crosslinked particle comprising gelatin and an essential oil, wherein at least a portion of the gelatin is crosslinked.

2. The crosslinked particle of claim 1, wherein the particle is a nanoparticle.

3. The crosslinked particle of claim 1, wherein the essential oil is selected from the group consisting of peppermint oil, oregano oil, thymol, menthol, methyl salicylate, eucalyptol, carvacrol, carvacrol methyl ether, camphor, anethole, carvone, eugenol, isoeugenol, limonene, osimen, n-decyl alcohol, citronel, a-salpineol, methyl acetate, citronellyl acetate, methyl eugenol, cineol, linalool, ethyl linalaol, safrola vanillin, spearmint oil, lemon oil, orange oil, sage oil, rosemary oil, cinnamon oil, pimento oil, laurel oil, cedar leaf oil, clove oil, cilantro oil, coriander oil, or a combination thereof.

4. The crosslinked particle of claim 1, wherein the gelatin comprises a histidine residue and at least one of a hydroxyproline residue, a tyrosine residue, and a threonine residue.

5. The crosslinked particle of claim 4, wherein the gelatin comprises a histidine residue and a hydroxyproline residue, wherein the hydroxyproline residue and the histidine residue are present in a molar ratio of at least 2:1.

6. The crosslinked particle of claim 1, wherein the gelatin has a structure comprising the residues of formula (I) and formula (II)

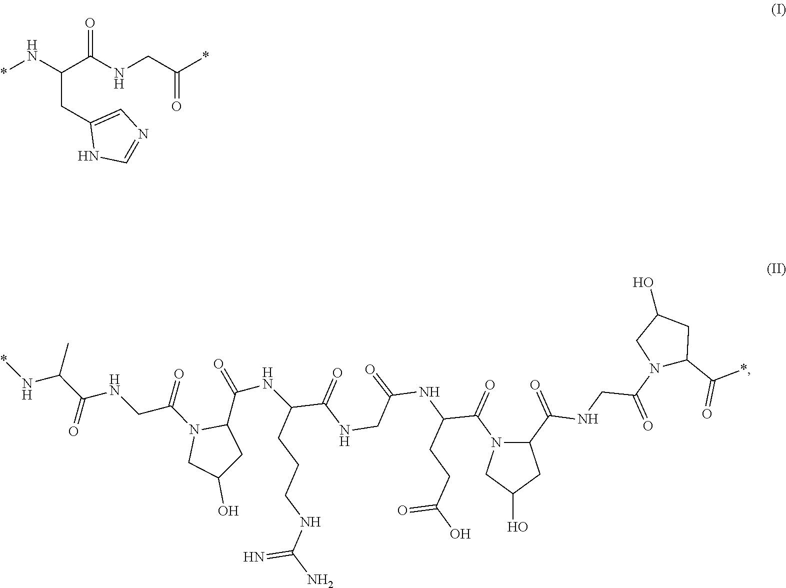

7. The crosslinked particle of claim 1, wherein the gelatin comprises Type B gelatin, Type A gelatin, or a combination thereof.

8. The crosslinked particle of claim 1, wherein the particle has a core-shell structure, wherein the core of the particle comprises the essential oil and gelatin that is at least partially crosslinked, and the shell comprises crosslinked gelatin.

9. The crosslinked particle of claim 1, wherein the gelatin is crosslinked using a photoinitiator.

10. The crosslinked particle of claim 1, wherein the crosslinked gelatin comprises one or more crosslinks of formula (III)

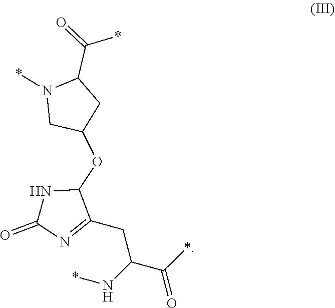

11. The crosslinked particle of claim 1, comprising:
90 to 99.9 weight percent of the essential oil; and
0.1 to 10 weight percent of the gelatin;
wherein weight percent is based on the total weight of the particle.

12. A composition comprising a plurality of crosslinked particles according to claim 1.

13. A method of making the crosslinked particles of claim 1, the method comprising:
combining an essential oil and an aqueous solution comprising gelatin to provide a mixture, wherein at least one of the essential oil or the aqueous solution comprises an initiator;
emulsifying the mixture to provide an intermediate composition comprising a plurality of particles comprising
a liquid hydrophobic core comprising an essential oil and gelatin; and
a shell encapsulating the core, the shell comprising the gelatin; and exposing the intermediate composition to conditions effective to crosslink the gelatin to provide the crosslinked particles.

14. The method of claim 13, wherein the conditions effective to crosslink the gelatin comprise exposing the intermediate composition to ultraviolet irradiation.

15. The method of claim 13, wherein the initiator comprises a photoinitiator.

16. A method of treating a bacterial or a fungal infection, the method comprising contacting the composition of claim 12 with a bacterial infection or a fungal infection.

17. The method of claim 16, comprising contacting the composition of claim 12 with a bacterial biofilm, wherein the bacterial infection comprises Escherichia coli, Pseudomonas bacteria, Staphylococcal bacteria, Enterobacteriaceae bacteria, Streptococcus bacteria, Haemophilus influenzae, Leptospira interrogans, Legionella bacteria, Mycobacterium tuberculosis, Candida albicans bacteria, Acinetobacter baumannii bacteria, Stenotrophomonas maltophilia bacteria, Clostridium difficile bacteria, Enterococcus bacteria, Klebsiella pneumoniae bacteria, Necrotizing fascitis bacteria, Corynebacterium bacteria, Helicobacter pylori bacteria, Campylobacter bacteria, Salmonellae bacteria, Neisseria gonorrhoeae bacteria, Haemophilus influenza bacteria, Shigella bacteria, or a combination thereof.

18. The method of claim 16, comprising contacting the composition of claim 12 with a fungal infection, wherein the fungal infection comprises Alternaria alternata, Aspergillus fumigatus, Aspergillus niger, Aspergillus flavus, Aspergillus nidulans, Aspergillus paraciticus, Candida albicans, Candida dubliniensis, Candida famata, Candida glabrata, Candida Candida haemdionii, Candida kejyr, Candida krusei, Candida lusitaniae, Candida norvegensis, Candida parapsilosis, Candida tropicalis, Candida viswanathii, Epidermophyton floccosum, Fusarium graminearum, Fusarium oxysporum, Fusarium solani, Fusarium monolifortne, Trychophyton rubrum, Trychophyton nientagrophytes, Trychophyton inter digitales, Trychophyton tonsurans, Cryptococcus neoformans, Cryptococcus gattii, Cryptococcus grubii, Colletotrichum graminicola, Microsporum canis, Microsporarn gypseum, marneffei, Tricosporon Trichosporon asahii, Trichosporon inkin, Trichosporon asteroides, Trichosporon cutaneum, Trichosporon domesticum, Trichosporon mucoides, Trichosporon ovoides, Trichosporon pullulans, Trichosporon loubieri, Trichosporon japonicum Scedosporiurn apiospermum, Scedosporium prolifwans, Paecilomyces variotn, Paecilornyces lilacinus, Acremonium stricutm, Cladophialophora bantiana, Wangiella dermatitidis, Ramichioridium obovoideum, Chaetomium atrobrunneum, Dactiaria gaillopavum, Bipolaris spp, Exserohilum rostratum, Absidia corymbifera, Apophysomyces elegans, Mucor indicus, Rhizomucor pusillus, Rhizopus oryzac, Cunninghamellia bertholletiae, Cokeromyces recurvatus, Saksenaea vasiformis, Syncephalastrum racemosum, Basidiobolus ranarum, Conidiobolus coronatuslConidiobolus incongruus, Blastomyces dermatitidis, Coccidioides Coccidioides posadasii, Histoplasma capsulatum, Paracoccidioides brasiliensis, Pseudalleschcmia Sporothrix schenckii, Alternaria brassicicola, Alternaria alternata, Aspergillus nidulans, Botrytis cinerea, Cercospora beticola, Cercospora zeae maydis, Cochliobolus heterostrophus, Exserohilum turcicum, Fusarium cuimorum, Fusarium oxysporum, Fusarium oxysporurn f. sp. dianthi, Fusarium solani, Fusarium pseudograminearum, Fusarium verticilloides, Gaeumannomyces graminis var. tritici, Piasmodiophora brassicae, Scierotinia scierotiorum, Stenocarpella (Diplodia) maydis, Thielaviopsis hasicola, Verticillium dahliae, Ustilago zeae, Puccinia sorghi, Macrophornina phaseolina, Phialophora gregata, Diaporthe phaseolorum, Cercospora sojina, Phytophthora sojae, Rhizoctonia solani, Phakopsora pachyrhizi, Alternaria macrospora, Cercospora gossypina, Phoma exigua, Puccinia schedonnardii, Puccinia cacabata, Phymatotrichopsis omnivora, Fusarium avenaceum, Alternaria brassicae, Alternaria raphani, Erysiphe graminis (Biumena graminis), Septoria trifici, Septoria nodorum, Mycosphaerelia zeae, Rhizoctonia cerealis, Ustilago trifici, Puccinia graminis, Puccinia triticina, Tilletia indica, Tilletia caries, Tillietia controversa, Alternaria solani, Alternaria brassicae, Alternaria brassicola, Monolinia fructicola, Venturia inaequalis, Ciadosporum carpophilurn, Botryosphaeria obtuse, vaccinia-corymbosi, Scierotinia homoeocarpa, Podosphaera xanthii, Podosphaera fuliginea, Erysiphe cichoracearum, Biumeria graminis f. sp. Tritici, biumeria graminis f. sp. Hordei, Microsphaera diffusa, Erysiphe necator, Leveillula Taurica, Podosphaera leucotricha, Podosphaera aphanis, Sawadaea tulasnei, Erysiphe berberidis, Golovinomyces orontii, Peronospora belbahrii, Pseudoperonospora cubensis, Plasmopara Pseudoperonospora humuli, Peronospora manshurica, Plasraopara halstedii, Phytopthora capcisi, Phytopthora infestans, Phytopthora cinnamomic, Phytopthora sojae, Phytopthora agathidicida, Phytopthora cactorum, Phytopthora citricola, Phytopthora fragariae, Phytopthora kernoviae, Phytopthora lateralis, Phytopthora megakarya, Phytopthora multivora, Phytopthora nicotianae, Phytopthora palmivora, Phytopthora ramorum, Phytopthora quercina, or a combination thereof.
